(19) United States
(12) Patent Application Publication
(10) Pub. No.: US 2024/0042128 A1
(43) Pub. Date: Feb. 8, 2024

(54) LIQUID MEDICINE INJECTION DEVICE

(71) Applicant: EOFLOW CO., LTD., Seongnam-si, Gyeonggi-do (KR)

(72) Inventors: Won Kyung Bang, Seoul (KR); Seung Ha Kim, Goyang-si, Gyeonggi-do (KR); Yongjoon Han, Seoul (KR)

(21) Appl. No.: 17/754,471

(22) PCT Filed: Sep. 29, 2020
(86) PCT No.: PCT/KR2020/013407

§ 371 Date: Apr. 1, 2022

(30) Foreign Application Priority Data
Oct. 4, 2019 (KR) 10-2019-0123365
Oct. 4, 2019 (KR) 10-2019-0123366

(51) Int. Cl. A61M 5/145; A61M 5/142

(52) U.S. Cl. CPC A61M 5/1452; A61M 5/14248; A61M 2205/8206

(57) ABSTRACT

Provided is a drug injection device including: a base body; a needle assembly mounted on the base body; a reservoir mounted on the base body and fluidly connected with the needle assembly; a driving unit including a rod that is engaged with a plunger disposed inside the reservoir and moves along the reservoir, and a driving wheel that transmits a driving force to the rod; a clutch unit disposed between the rod and the driving wheel; and a trigger member rotatably disposed on one side of the base body and coupling the rod with the driving wheel by pressing the clutch unit.

### TECHNICAL FIELD

The present disclosure relates to a drug injection device.

### BACKGROUND ART

In general, drug injection devices such as insulin injection devices are used to inject drugs into patients' bodies. Although drug injection devices are sometimes used by professional medical staff such as doctors or nurses, in most cases, it is used by general public such as the patients themselves or guardians.

Diabetic patients, especially pediatric diabetic patients, need to inject drugs such as insulin into the body at regular intervals. Therefore, a patch-type drug injection device that is used by being attached to human body for a certain period of time, is being developed, and this type of drug injection device may be used while being attached to the body such as the patient's abdomen or waist in a patch-type for a certain period of time.

In order to increase the effect through drug injection, the drug injection device needs to be controlled to precisely inject the drug into the patient's body, and it is important to precisely inject a small amount of the drug through small drug injection devices.

In the case of being attached to the human body, drug injection devices need to have excellent wearability, ease of use, excellent durability, and low power consumption. In particular, since the drug injection device is directly attached to the skin of the patient and used, it is important for the user to conveniently and safely operate the drug injection device.

### DESCRIPTION OF EMBODIMENTS

### Technical Problem

The present disclosure provides a drug delivery device that is safely operated and is capable of delivering drugs accurately.

### Technical Solution to Problem

An aspect of the present disclosure provides a drug injection device including: a base body; a needle assembly mounted on the base body; a reservoir mounted on the base body and fluidly connected with the needle assembly; a driving unit including a rod that is engaged with a plunger disposed inside the reservoir and moves along the reservoir, and a driving wheel that transmits a driving force to the rod; a clutch unit disposed between the rod and the driving wheel; and a trigger member rotatably disposed on one side of the base body and coupling the rod with the driving wheel by pressing the clutch unit.

### Advantageous Effects of Disclosure

The drug injection device and the method of operating the same, according to the present disclosure, can be operated simply and safely by the user. When the user rotates the sleeve of the needle assembly, the cannula of the needle assembly is inserted into the object, thereby completing the preparation process for the injection of a drug. Together with the completion, the clutch unit may engage the driving unit with the driving module to allow the drug to be delivered from the reservoir into the needle, and at the same time, the driving of the driving module is started. Therefore, the user can safely start driving of the drug injection device by simply rotating the needle assembly.

In relation to the drug injection device and the method of driving the same according to the present disclosure, after the cannula of the needle assembly is inserted into the object, the structure for discharging the drug is drivably engaged by the clutch unit. Accordingly, the drug may be stably injected in a preset amount into the object.

In relation to the drug injection device and the method of driving the same according to the present disclosure, the driving of the drug injection device is intuitively started by rotation of the needle assembly. Also, since the rotation directions of the needle assembly and the trigger member are limited to one direction, the rotation distance is limited to a preset distance. Accordingly, user safety can be secured.

In relation to the drug injection device and the method of driving the same according to the present disclosure, the drug may be discharged in a preset amount since the driving module is driven in an optimized driving environment. The starter may move the position of the driving shaft before the driving module is driven, so that the case in which the contact member is attached on the inner surface of the driving module to interrupt the driving of the driving module, may be prevented. Since the storability of the drug injection device is improved and precision thereof is maintained, even when the drug injection device is driven after being stored for a long time, the driving module may be operated smoothly at the beginning, so that the drug can be discharged in a preset amount to the object. The scope of the present disclosure is not limited by these effects.

### BRIEF DESCRIPTION OF DRAWINGS

FIG. 1 is a perspective view illustrating a drug injection device according to an embodiment of the present disclosure.

FIG. 2 is a perspective view for describing an internal arrangement of the drug injection device of FIG. 1.

FIG. 3 is a plan view showing the drug injection device of FIG. 2.

FIG. 4 is an enlarged view of area A of FIG. 3.

FIGS. 5A to 5C are cross-sectional views illustrating the operation of a needle assembly of FIG. 2.

FIG. 6 is a perspective view of a partial configuration of FIG. 3.

FIG. 7 is a perspective view illustrating that a trigger member of FIG. 6 is rotated to drive a clutch unit.

FIG. 8 is a cross-sectional view illustrating a coupling relationship between a reservoir and the clutch unit of FIG. 6.

FIG. 9 is a cross-sectional view illustrating a coupling relationship between a reservoir and the clutch unit of FIG. 7.

FIG. 10 is a view showing the arrangement relationship between a driving wheel and the clutch unit of FIG. 6.

FIG. 11 is a view showing the arrangement relationship between a driving wheel and the clutch unit of FIG. 7.

FIGS. 12A to 12C are diagrams illustrating the driving of a driving module according to the rotation of the trigger member of FIG. 2 from one side.

FIGS. 13A to 13C are diagrams illustrating the driving of a driving module according to the rotation of the trigger member of FIG. 2 from another side.

FIG. 14 is a flowchart illustrating a method of driving a drug injection device according to another embodiment of the present disclosure.

FIG. 15 is a flowchart illustrating some operations of FIG. 14.

### BEST MODE

An aspect of the present disclosure provides a drug injection device including: a base body; a needle assembly mounted on the base body; a reservoir mounted on the base body and fluidly connected with the needle assembly; a driving unit including a rod that is engaged with a plunger disposed inside the reservoir and moves along the reservoir, and a driving wheel that transmits a driving force to the rod; a clutch unit disposed between the rod and the driving wheel; and a trigger member rotatably disposed on one side of the base body and coupling the rod with the driving wheel by pressing the clutch unit.

In an embodiment, the needle assembly rotates in one direction to press an end of the trigger member so as to rotate the trigger member, and due to the rotation of the trigger member, another end of the trigger member presses a coupler of the clutch unit so that an end of the coupler is separated from the driving wheel.

In an embodiment, the trigger member may press the clutch unit after a cannula of the needle assembly is inserted.

In an embodiment, the base body may be provided with a stopper for limiting the rotation distance of the trigger member.

In embodiment, the clutch unit may include: an inserter inserted into the rod; and a coupler disposed on the outside of the inserter, and having an end that is detachably mounted on the driving wheel.

In an embodiment, the driving wheel includes: a first supporter disposed on one surface of the driving wheel to support an end of the coupler so as to maintain a state in which the diameter of the coupler is expanded; and a second supporter disposed to be spaced apart from the first supporter on the surface of the driving wheel and supporting the end of the coupler so as to maintain a state in which the diameter of the coupler is contracted.

Another aspect of the present disclosure has a drug injection device including: a base body, a needle assembly mounted on the base body, a reservoir mounted on the base body, and having an inner space in which a plunger is disposed, a driving module having a driving shaft, and generating a driving force for moving the plunger, a trigger member rotatably disposed on one side of the base body, wherein due to rotation, the contact thereof at an end of the driving shaft is released to start the driving of the driving module.

In an embodiment, the needle assembly rotates in one direction to press an end of the trigger member so as to rotate the trigger member, and due to the rotation of the trigger member, another end of the trigger member may be separated from an end of the driving axis.

In embodiment, the drug injection device may further include a starter mounted on the base body and arranged to support the end of the trigger member, wherein due to the rotation of the trigger member, the starter presses the end of the driving shaft.

In addition, the driving module includes a contact member mounted on the driving shaft and being in contact with the inner surface of the housing, and the starter presses the end of the driving shaft to linearly move the driving shaft and the contact member.

In an embodiment, the contact member may be moved on the inner surface of the housing before the driving module is driven.

In an embodiment, the base body may be provided with a stopper for limiting the rotation distance of the trigger member.

### MODE OF DISCLOSURE

Since the present disclosure may undergo various transformations and may have various embodiments, specific embodiments are illustrated in the drawings and described in detail in the detailed description. Effects and features of the present disclosure, and a method of achieving them will become apparent with reference to the embodiments described below in detail in conjunction with the drawings. However, the present disclosure is not limited to the embodiments disclosed below and may be implemented in various forms.

Hereinafter, embodiments of the present disclosure will be described in detail with reference to the accompanying drawings, and when described with reference to the drawings, the same or corresponding components are given the same reference numerals, and the overlapping description thereof will be omitted.

In the following examples, the singular expression includes the plural expression unless the context clearly dictates otherwise.

In the following embodiments, terms such as “include” or “have” refer to that the features or components described in the specification are present, and the possibility that one or more other features or components will be added is not excluded in advance.

In cases where certain embodiments are able to be implemented otherwise, a specific process sequence may be performed different from the described sequence. For example, two processes described in succession may be performed substantially simultaneously, or may be performed in an order opposite to the order described.

In the drawings, the size of the components may be exaggerated or reduced for convenience of description. For example, since the size and thickness of each component shown in the drawings are arbitrarily indicated for convenience of description, the embodiments to be described hereinafter are not necessarily limited to what is illustrated.

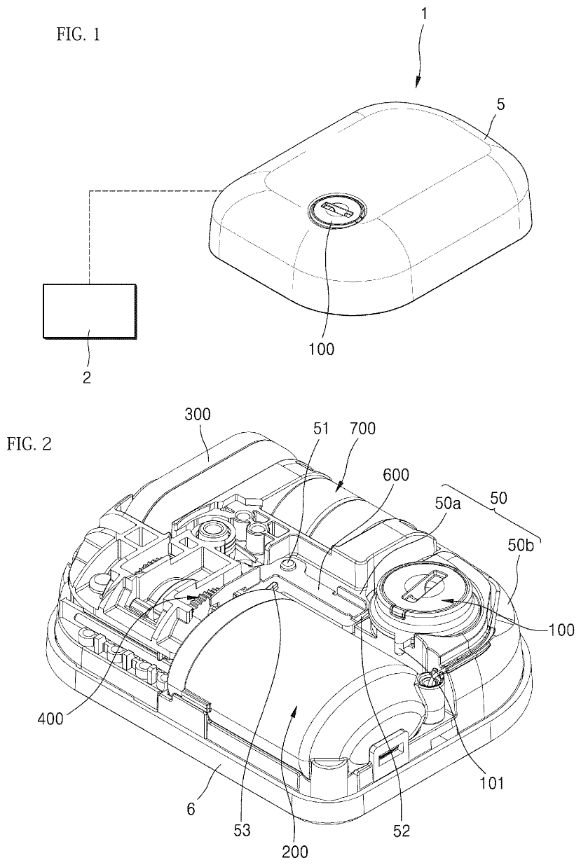

FIG. 1 is a perspective view illustrating a drug injection device 1 according to an embodiment of the present disclosure.

Referring to FIG. 1, a drug injection device 1 is attached to an object to which a drug is to be injected, etc., and may inject a preset amount of a drug stored therein to the user. As an optional embodiment, the drug injection device 1 may be mounted on the user's body. In another optional embodiment, the drug injection device 1 may be mounted on an animal to inject a drug.

The drug injection device 1 may be used for various purposes depending on the type of drug to be injected. For example, the drug may include an insulin-based drug for diabetic patients, and may include other drugs for the pancreas, drugs for the heart, and other various types of drugs.

The drug injection device 1 may be connected to a remote device 2 connected by wire or wirelessly. The user may use the drug injection device 1 by operating the remote device 2, and may monitor the usage state of the drug injection device 1. For example, the amount of drug injected from drug injection device 1, the number of injections of the drug, the amount of drug stored in the reservoir 200, the user's bio information, etc. may be monitored, and based on the monitoring information, the user may drive the drug injection device 1.

In an embodiment, the remote device 2 refers to a communication terminal capable of using an application in a wired/wireless communication environment. Here, the remote device 2 may be the user's portable terminal. In more detail, the remote device 2 may include computers (e.g., desktop, laptop, tablet, etc.), media computing platforms (e.g., cable, satellite set-top boxes, digital video recorders), handheld computing devices (e.g., PDAs, email clients, etc.), any types of hand phones, wearable devices that may be attached to or mounted on users' bodies, different kinds of computing, or any types of communication platforms, but the present disclosure is not limited thereto.

The drug injection device 1 and the remote device 2 may communicate through a communication network. In this case, the communication network refers to a communication network that provides a connection path so that the remote device 2 may transmit and receive data after accessing a service server (not illustrated). The communication network may include, for example, a wired network such as local area networks (LANs), wide area networks (WANs), metropolitan area networks (MANs), integrated service digital networks (ISDNs), or a wireless network such as wireless LANs, code division multiple access (CDMA), Bluetooth, and satellite communications, but the scope of the present disclosure is not limited thereto.

According to FIG. 1, the remote device 2 is illustrated as a single device, but the present disclosure is not necessarily limited thereto, and may include a plurality of devices capable of communicating with the drug injection device 1.

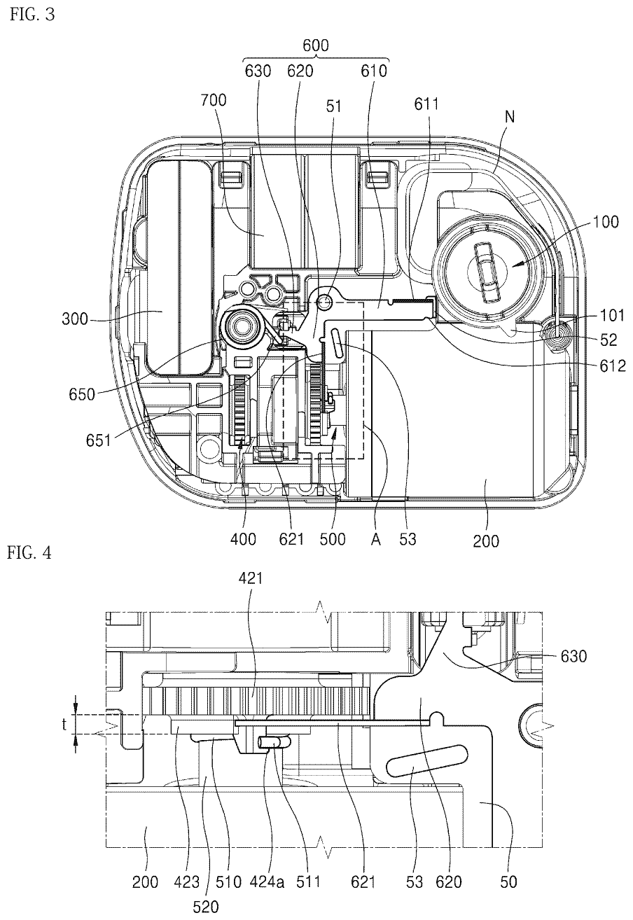

FIG. 2 is a perspective view for describing an internal arrangement of an example of the drug injection device 1 of FIG. 1, FIG. 3 is a plan view showing the drug injection device 1 of FIG. 2, and FIG. 4 is an enlarged view of area A of FIG. 3.

Referring to FIGS. 1 to 4, an embodiment of the drug injection device 1 may include a housing 5 covering the outside thereof, and an attachment portion 6 positioned adjacent to the user's skin. The drug injection device 1 may include a plurality of components disposed in an interior space between the housing 5 and the attachment portion 6. A separate bonding member may be further disposed between the attachment portion 6 and the user's skin, and the drug injection device 1 may be fixed on the skin by the bonding member.

The drug injection device 1 may include a base body 50, a needle assembly 100, a reservoir 200, a driving module 300, a driving unit 400, a clutch unit 500, a trigger member 600, and a battery 700.

The base body 50 forms the basic frame of the housing 5 and is mounted in the interior space of the housing 5. A plurality of base bodies 50 may be provided. In an embodiment, a first body 50a that covers upper sides of the internal parts and a second body 50b that covers lower sides of the internal parts, may be provided. The first body 50a and the second body 50b are assembled to fix the internal parts of the drug injection device 1 to a preset position. In another embodiment, the base body may be formed as an integral single frame.

The base body 50 can provide a space for the trigger member 600 to rotate. The base body 50 supports the trigger member 600, and the trigger member 600 may rotate based on an rotation axis 51 protruding from the base body 50.

The base body 50 may include a stopper that limits the rotation distance of the trigger member 600. A plurality of stoppers may be provided, and the movement distance of the trigger member 600 may be limited so that the trigger member 600 rotates to a preset point. According to an embodiment, the stopper may include a first stopper 52 and a second stopper 53.

The first stopper 52 protrudes upward from the base body 50 and is disposed adjacent to the needle assembly 100. The first stopper 52 may be arranged to be in contact with a first end 610 of the trigger member 600, and may limit the rotation direction and the rotation distance so that the first end 610 does not rotate the opposite direction after rotating in one direction.

In detail, the first stopper 52 may have an upper surface 52a formed such that a portion thereof in contact with the trigger member 600 protrudes obliquely (see FIG. 6). When the first end 610 of the trigger member 600 rotates in one direction, the first end 610 moves along the upper surface 52a of the first stopper 52. Since the upper surface 52a of the first stopper 52 guides the movement of the trigger member 600, the rotation of the needle assembly 100 may lead to rotation of the trigger member 600 smoothly.

The first stopper 52 may have a side wall 52b that limits the rotation direction of the trigger member 600. The side wall 52b may extend from the upper surface 52a and be formed substantially perpendicular to the plane of the base body 50. The side wall 52b prevents, after the trigger member 600 rotates by a preset rotation distance in one direction, the needle assembly 100 and the trigger member 600 from rotating in the opposite direction to ensure the stability of the drug injection device 1.

The second stopper 53 may be arranged adjacent to the reservoir 200, the driving unit 400, and the clutch unit 500. The second stopper 53 may be disposed to protrude from the upper portion of the base body 50 to limit the movement distance of a second end 620 of the trigger member 600. In an embodiment, the second stopper 53 may have a longitudinal extension line passing through the center of the rotation axis 51.

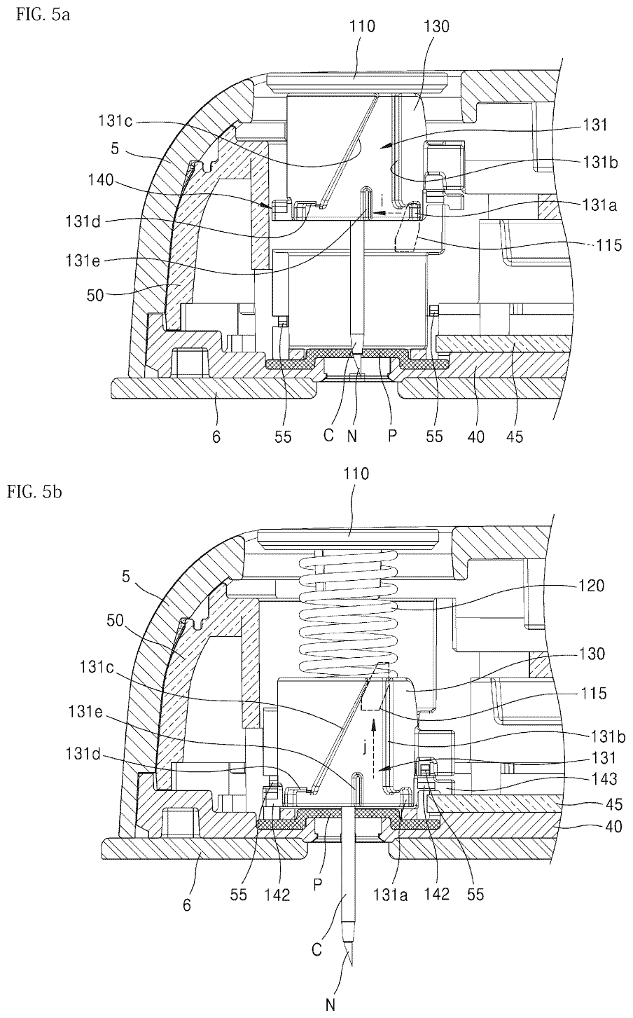

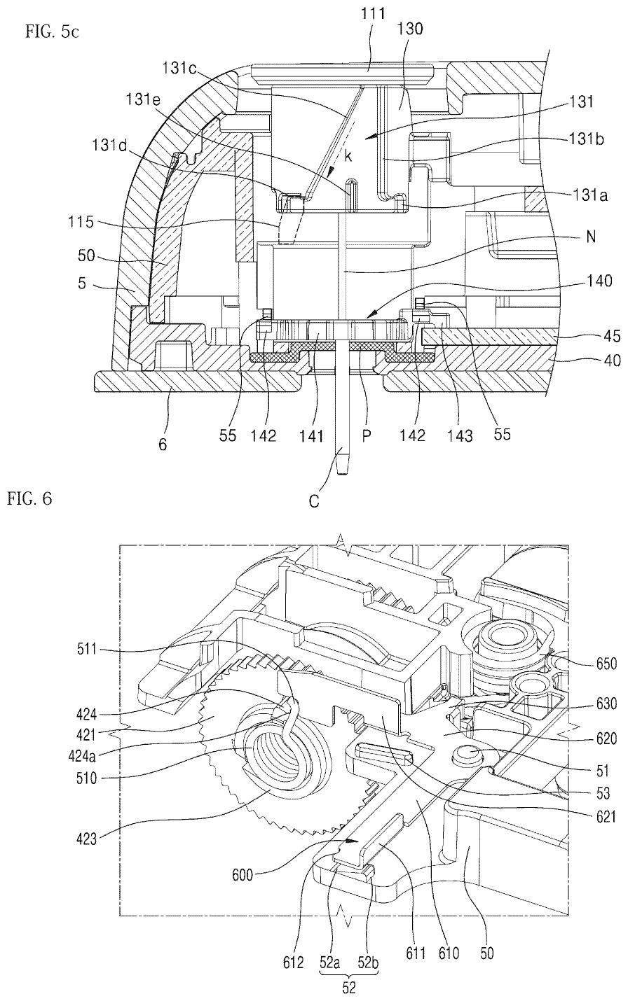

FIGS. 5A to 5C are cross-sectional views illustrating the driving of the needle assembly 100 according to an embodiment.

Referring to FIGS. 5A to 5C, the needle assembly 100 may be mounted on the base body 50. Due to the rotation of the needle assembly 100, a needle (N) and/or a cannula (C) may be moved in the axial direction. The needle assembly 100 may include a sleeve 110, an elastic member 120, a first holder 130, a second holder 140, a needle N, a cannula C, and a patch P.

The sleeve 110 forms the exterior of the needle assembly 100 and is rotatable about the longitudinal axis thereof as a central axis. An elastic member 120 is disposed inside the sleeve 110, and the sleeve 110 may receive an expansion force from the elastic member 120.

A knob 101 may be disposed on the outside of the sleeve 110. When the needle assembly 100 rotates, the knob 101 may press the first end 610 of the trigger member 600 to rotate the trigger member 600.

A moving protrusion 115 may be disposed on the inner surface of the sleeve 110. The moving protrusion 115 may move along a guide groove 131 disposed on the outer peripheral surface of the first holder 130. As the sleeve 110 rotates, the moving protrusion 115 moves along the sidewalls of the guide groove 131 so that the first holder 130 may be raised or lowered.

The elastic member 120 may be disposed between the sleeve 110 and the first holder 130. When the elastic member 120 expands, the first holder 130 may be moved downward. In addition, when the first holder 130 moves upward, the elastic member 120 may be compressed.

The first holder 130 may support the needle N. Since the needle N is inserted and fixed on one side of the first holder 130, when the first holder 130 moves in the axial direction, the needle N also moves together with the first holder 130. The first holder 130 is arranged in the inner space of the sleeve 110, and the elastic member 120 may be arranged on an upper portion of the first holder 130.

The first holder 130 may have a guide groove 131 on the outer surface thereof. The guide groove 131 may guide the movement of the moving protrusion 115. When the sleeve 110 is rotated, the moving protrusion 115 moves along the preset path formed by the guide groove 131, so that the first holder 130 may be raised or lowered.

The guide groove 131 may include a first stop groove 131a, an elevation groove 131b, an inclined groove 131c, a second stop groove 131d, and a movement limiting groove 131e. The first stop groove 131a is a groove in which the moving protrusion 115 is initially located, and at this position, the elastic member 120 maintains the contracted state. The elevation groove 131b extends in the longitudinal direction of the first holder 130. When the elastic member 120 expands, the first holder 130 descends and the moving protrusion 115 moves along the elevation groove 131b. The inclined groove 131c is extended to have a preset slope. When the moving protrusion 115 moves along the inclined groove 131c, the elastic member 120 contracts again and the first holder 130 is raised again. The second stop groove 131d is a groove where the moving protrusion 115 returns to its initial position, and at this time, the elastic member 120 maintains the contracted state again.

The second holder 140 is disposed to face one side of the first holder 130 and may support the cannula C. The second holder 140 is formed of a flexible material, and when an external force is applied thereto, the shape thereof may be instantaneously changed. In another embodiment, the second holder 140 has a certain level of rigidity and may be instantaneously deformed in shape by an external force.

The second holder 140 maintains being contact with the first holder 130 in the positions illustrated in FIGS. 5A and 5B, but when the elastic member 120 contracts again as shown in FIG. 5C, the second holder 140 is separated from the first holder 130 and fixed on the base body 50.

The cannula C is inserted into the center of a central body 141, and the needle N may be selectively coupled according to the movement of the first holder 130. The central body 141 may contact the lower end of the first holder 130.

A locking protrusion 142 may protrude outward from the central body 141 and may be formed to be flexible. A plurality of locking protrusions 142 may be provided. Referring to FIG. 5C, when the second holder 140 is lowered, the locking protrusion 142 may be fixed on a ledge part 55, and thus, even when the first holder 130 is raised again, the second holder 140 may be fixed on the base body 50.

A sensor 143 may be disposed on one side of the locking protrusion 142. However, the present disclosure is not limited thereto, and the sensor 143 may be disposed on the outer surface of the central body 141. The sensor 143 may detect whether the cannula C has been correctly inserted into the user's skin. The sensor 143 may sense a contact between the second holder 140 and a base 40.

In detail, since the second holder 140 moves beyond the ledge part 55 when moving downwards, the sensor 143 may identify the contact with a contact portion 45 disposed on the base 40. The sensor 143 is formed of a conductive material, and thus, when comes into contact with the contact portion 45, an electrical signal is generated, so that a controller (not shown) may identify whether the second holder 140 has been inserted.

Since the needle N is fixed on the first holder 130, the needle N can be inserted or released from the cannula C by the axial movement of the first holder 130. An end of the needle N is connected to the reservoir 200 to allow the drug to be delivered, and another end thereof is inserted into the cannula C to move along the cannula C.

Since the cannula C is fixed on the second holder 140, the cannula C may be inserted into the user's skin by the axial movement of the second holder 140. Since the cannula C has a conduit shape that allow the needle N to be housed therein, the drug discharged from needle N may be injected into the user.

The patch P may be supported by the base 40, and may fix the position of cannula C. Since the end of the cannula C is supported by the patch P, it is possible to prevent the separation of the cannula C during storage or transport.

As shown in FIG. 5A, the needle N and the cannula C are initially disposed inside the needle assembly 100. When the user rotates the sleeve 110 first, the moving protrusion 115 moves in an i direction and moves beyond the first stop groove 131a.

As shown in FIG. 5B, by the expansion force of the elastic member 120, the first holder 130 may be lowered. The moving protrusion 115 does not actually move in the axial direction, but due to the lowering of the first holder 130, the moving protrusion 115 moves in a j direction along the elevation groove 131b relative to the first holder 130. The first holder 130 and the second holder 140 are lowered together, and the needle N and the cannula C pass through the patch P and are inserted into an object.

Referring to FIG. 5C, when the user rotates the sleeve 110 again, the moving protrusion 115 moves along the inclined groove 131c in a k direction and is inserted into the second stop groove 131d. Although the second holder 140 is fixed by the ledge part 55, the first holder 130 is raised according to the rotation of the sleeve 110, and the elastic member 120 re-contacts by the rise of the first holder 130.

At this time, the cannula C remains as being inserted into the user's skin, and the needle N is raised and separated from the object. However, the cannula C and the needle N are fluidly connected, and the drug injected from reservoir 200 may be injected into the user through the needle N and the cannula C.

The drug injection device 1 allows the user to simply rotate the needle assembly 100 so as to insert the cannula C into the object and start the injection of drug. The drug injection device 1 may be driven in such a manner that the user rotates the sleeve 110 to insert the cannula C into the object first, and the knob 101 presses the trigger member 600. In an embodiment, in a state in which the needle N is withdrawn from the object and only the cannula C is inserted into the object as shown in FIG. 5C, the knob 101 presses the trigger member 600 to drive the driving module 300 to deliver a drug, and the drug may be discharged in a preset amount from the reservoir 200. Therefore, the user may use the drug injection device 1 conveniently and stably.

Referring back to FIGS. 2 and 3, the reservoir 200 is mounted on the base body 50 and is connected to the needle assembly 100. The reservoir 200 may have a space for storing a drug, the space defined by a barrel 210 and a cap cover 220 (see FIG. 8). Inside the reservoir 200, a plunger 230 moves linearly to discharge the drug to the needle N.

The plunger 230 may be provided with a sealing portion 240 in a portion thereof in contact with the inner wall of the barrel 210 so as to prevent drug leakage during the movement of the plunger 230.

The driving module 300 may generate a driving force and transmit the driving force to the driving unit 400. Due to the driving force transmitted by the driving unit 400, the plunger 230 is linearly moved inside the reservoir 200 to discharge the drug.

When the driving units 400 are engaged with each other by the clutch unit 500, the driving module 300 rotates a driving wheel 420 of the driving unit 400, and due to the rotation of the driving wheel 420, a rod 410 is linearly moved so that the plunger 230 may move.

The driving module 300 may include a membrane 320 disposed inside a cover 310. The membrane 320 may partition the inner space of the driving module 300 into a first space S1 and a second space S2. The driving module 300 may linearly move a driving shaft 330 by a volume change in the first space S1 and the second space S2.

The membrane 320 may have a porous structure in which fluid and ions may move. The membrane 320 may be, for example, a frit-type membrane prepared by thermally calcining spherical silica. For example, the spherical silica used to form the membrane 320 may have a diameter of about 20 nm to about 500 nm, a diameter of about 30 nm to about 300 nm, or a diameter of about 40 nm to about 200 nm. When the diameter of the spherical silica satisfies the above-mentioned ranges, pressure due to the first fluid passing through the membrane 320, that is, pressure sufficient to move the driving shaft 330 may be generated.

Although it has been described that the membrane 320 includes spherical silica in the above-described embodiment, the membrane 320 is not limited thereto. In another embodiment, the membrane 320 may include any material that may cause an electrokinetic phenomenon by zetapotential, such as porous silica or porous alumina.

The membrane 320 may have a thickness of about 20 μm to about 10 mm, a thickness of about 300 μm to about 5 mm, or a thickness of about 1,000 μm to about 4 mm.

A first electrode body 321 and a second electrode body 322 may be disposed on both sides of the membrane 320, respectively. Each of the first electrode body 321 and the second electrode body 322 may have a porous plate shape.

The porous plate may be disposed to be in contact the main surfaces of the membrane 320 on both sides thereof, respectively. The porous plate may effectively move fluids and ions through the porous structure. The porous plate may have a structure in which an electrochemical reaction material is formed on a porous base layer. The electrochemical reaction material may be formed by, for example, electrodeposition or coating on the porous base layer through a method such as electroless plating, vacuum deposition, coating, or a sol-gel process.

The porous base layer may have a pore size of about 0.1 μm to about 500 μm, a pore size of about 5 μm to about 300 μm, or a pore size of about 10 μm to about 200 μm. When the pore size of the porous base layer satisfies the above-mentioned ranges, it is possible to effectively move the fluid and ions, thereby improving the stability, lifespan characteristics, and efficiency of the driving module 300.

The driving shaft 330 is disposed on one side of the cover 310 and may move linearly according to the volume change of the first space S1 and the second space S2. When a redox reaction occurs in the first electrode body 321 and the second electrode body 322, a volume change may occur in the first space S1 and the second space S2 by products (for example, hydrogen ions, water) generated by the redox reaction. The volume change in the first space S1 and the second space S2 results in pressing the driving shaft 330 to linearly move the driving shaft.

A moving unit 340 is disposed at an end of the driving shaft 330 to transmit a driving force generated by the driving module 300 to the driving unit 400. The moving unit 340 may include a body 341 inserted into the driving shaft 330, a guide protrusion 342 protruding from an upper portion of the body 341, and a connection shaft 343 connected to the driving unit 400.

A side wall of the guide protrusion 342 facing the trigger member 600 or a starter 650, may be inclined. An inclined surface 342a may have a certain degree of angle with respect to the driving shaft 330.

The connection shaft 343 may be connected to a connector (not shown) of the driving unit 400. As the driving shaft 330 moves linearly, the connection shaft 343 also moves linearly, and the connector may be moved to drive the driving wheel 420.

A contact member 350 may be mounted outside the driving shaft 330. The second space S2 defined by the inner space of the driving module 300, for example, the inner surface of the cover 310 and one side of the driving shaft 330, may be an enclosed space, and a fluid exists therein. The contact member 350 may prevent fluid from leaking into the gap between the inner surface of the cover 310 and the driving shaft 330.

The contact member 350 may include a material having a certain level of elasticity to tightly seal the gap between the inner surface of the cover 310 and the driving shaft 330. In an embodiment, the contact member 350 may include a silicon-based and/or a rubber-based material.

All kinds of devices with drug suction power and drug discharge power by electricity may be used as the driving module 300. For example, all kinds of pumps such as mechanical displacement type micropumps and electromagnetic motion type micropumps may be used. The mechanical displacement micropump is a pump that uses a motion of a solid or fluid, such as a gear or diagram, to generate a pressure difference to induce a flow of fluid, and includes a diaphragm displacement pump, a fluid displacement pump, a rotary pump, and the like. The electromagnetic motion type micropump is a pump that directly uses electrical or magnetic energy to move a fluid, and includes an electro hydrodynamic pump (EHD), an electro osmotic pump, a magneto hydrodynamic pump, an electro wetting pump, and the like.

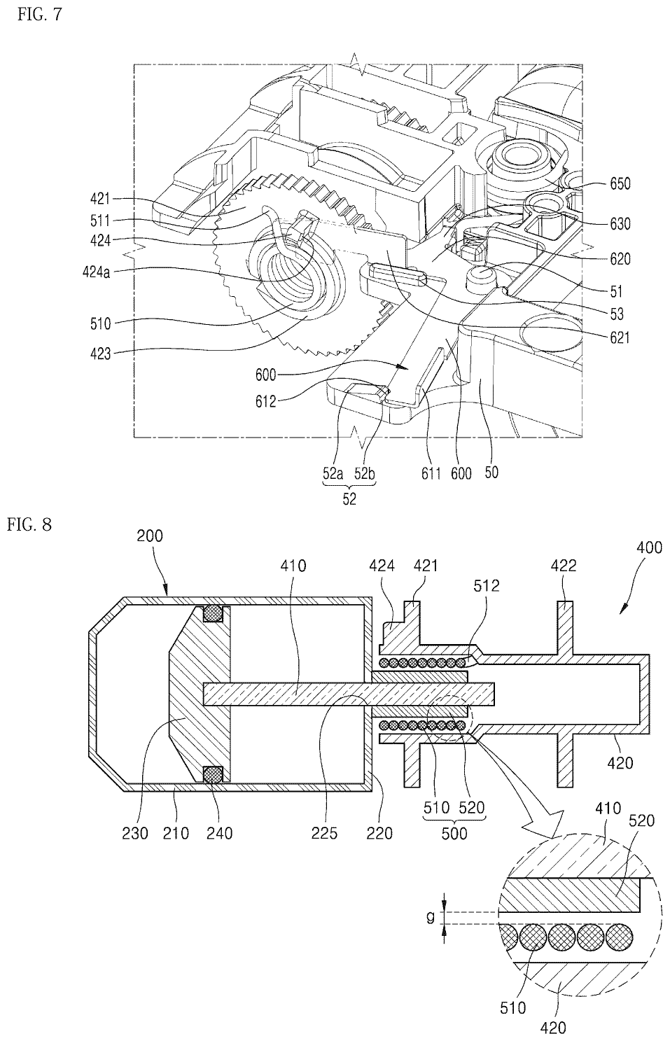

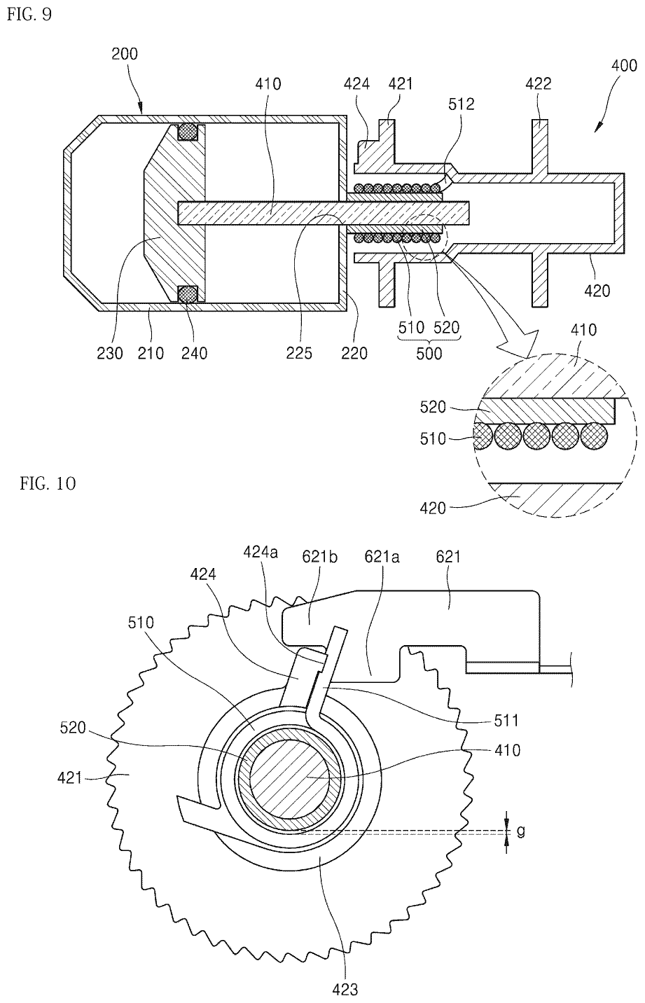

FIG. 6 is a perspective view of a partial configuration of FIG. 3, and FIG. 7 is a perspective view illustrating that the trigger member 600 of FIG. 6 is rotated to drive the clutch unit 500. FIG. 8 is a cross-sectional view illustrating a coupling relationship between the reservoir 200 and the clutch unit 500 of FIG. 6, and FIG. 9 is a cross-sectional view illustrating a coupling relationship between the reservoir 200 and the clutch unit 500 of FIG. 7.

Referring to FIGS. 6 to 9, the driving unit 400 may be provided between the driving module 300 and the reservoir 200, and may move the plunger 230 disposed in the reservoir 200 by a driving force generated by the driving module 300. However, the driving unit 400 may move the plunger 230 only when the rod 410 and the driving wheel 420 are coupled or connected to each other by the clutch unit 500.

The rod 410 may be connected to the plunger 230 and extends in one direction. The rod 410 may be inserted into an opening 225 of the cap cover 220, and the rod 410 may move in the longitudinal direction of the reservoir 200 to move the plunger 230.

The driving wheel 420 may be drivably connected to the driving module 300 and may be rotated by the driving of the driving module 300. The driving wheel 420 may have a first connection end 421 and a second connection end 422, and a space in which the rod 410 may move. At least one of the first connection end 421 and the second connection end 422 is continuously drivably connected to the driving module 300, so that the driving wheel 420 may be rotated by the driving of the driving module 300.

In an embodiment, the first connection end 421 and the second connection end 422 may have a gear teeth shape. A connector (not shown) connected to the driving module 300 may press the gear teeth to rotate the driving wheel 420.

The driving wheel 420 may include a hub 423 protruding from the first connection end 421. Referring to FIG. 4, the hub 423 may protrude to a preset height t. The hub 423 may form a space into which a second flange 621 of the trigger member 600 is inserted.

The driving wheel 420 may include a first supporter 424 that protrudes from one side thereof and supports an end 511 of the coupler 510. As shown in FIG. 6, the first supporter 424 may be disposed on one surface of the driving wheel 420 to support the end 511 of the coupler 510 so as to maintain the expanded diameter of the coupler 510.

As an optional embodiment, the first supporter 424 may have, on an end thereof, a protrusion 424a which protrudes to prevent the separation of the end 511 of the coupler 510.

When the coupler 510 is separated from the first supporter 424, the coupler 510 grips an inserter 520 while the diameter thereof is reduced. The coupler 510 retracts to a set radius so that the coupler 510 may provide a preset grip force to the inserter 520.

The clutch unit 500 may drivably connect the driving module 300 and the driving unit 400 to each other. The clutch unit 500 may be disposed between the rod 410 and the driving wheel 420, and may include the inserter 520 and the coupler 510.

The inserter 520 may be arranged such that at least a portion thereof is inserted into the rod 410. The inserter 520 may be disposed to cover the outside of the rod 410. The inserter 520 may connect the driving module 300 and the rod 410 according to the operation of the coupler 510.

In an embodiment, the rod 410 and the inserter 520 may have the form of a screw and the form of a thread, respectively. A thread is formed on the outer peripheral surface of the rod 410, and a thread is formed on the inner peripheral surface of the inserter 520, so that the rod 410 and the inserter 520 may be connected in a screw-coupled manner. When the coupler 510 presses the outer side of the inserter 520 as shown in FIG. 7, the rod 410 and the inserter 520 may be coupled to the driving wheel 420 by the coupler 510. At this time, the driving wheel 420 and the inserter 520 rotate together, so that the rod 410 may linearly move in one direction.

The coupler 510 is disposed on the outer side of the inserter 520, and when activated, the coupler 510 may connect the rod 410 and the driving wheel 420 to each other. The coupler 510 is a part that can press the outer side of the inserter 520 with an elastic force, and is not limited to a specific shape. However, hereinafter, for convenience of explanation, an embodiment in which the coupler 510 is type of a spring, will be described.

The end 511 of the coupler 510 may be detachably mounted on the driving wheel 420. The end 511 is supported by the first supporter 424, and may be separated beyond the protrusion 424a of the first supporter 424 by a pressing force of the second flange 621. The other end 512 of the coupler 510 may be fixed on the driving wheel 420.

Therefore, when the end 511 of the coupler 510 is mounted on the driving wheel 420, the coupler 510 is mounted only on the driving wheel 420, so that the rod 410 and the driving wheel 420 may be drivably separated. However, when the end 511 of the coupler 510 grips the inserter 520, the coupler 510 may drivably connect the rod 410 and the driving wheel 420 to each other.

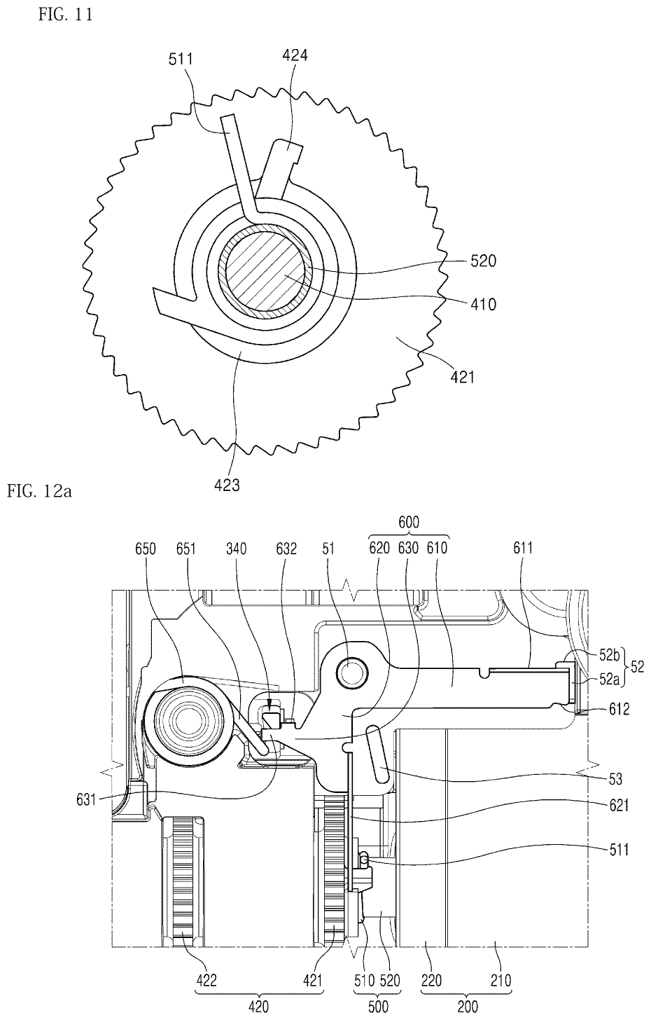

FIG. 10 is a view showing the arrangement relationship between the driving wheel 420 and the clutch unit 500 of FIG. 6, and FIG. 11 is a view showing the arrangement relationship between the driving wheel 420 and the clutch unit 500 of FIG. 7.

Referring to FIGS. 8 and 10, when the clutch unit 500 is not activated, when the drug is injected into the reservoir 200 for drug storage, the rod 410 and the inserter 520 may move in the inner space of the driving wheel 420. Since a gap of g is formed between the inserter 520 and the coupler 510, when the coupler 510 does not grip the inserter 520, the rod 410 and the inserter 520 may be allowed to move.

Referring to FIGS. 8 and 11, in the case where the clutch unit 500 is activated, when the driving wheel 420 rotates, the inserter 520 also rotates. At this time, since the rod 410 is coupled to the inserter 520, the rod 410 may linearly move forward by the rotation of the inserter 520.

The inserter 520 may have a certain level of flexibility so as to be engaged with the driving unit 400 by an elastic force applied by the coupler 510.

The trigger member 600 may generate a mechanical signal for the injection of the drug of drug injection device 1. The trigger member 600 is rotatably disposed on one side of the base body 50, and the trigger member 600 rotates to start driving of the driving module 300, and at the same time, the clutch unit 500 may drivably connect the driving unit 400.

The trigger member 600 may rotate in one direction around the rotation axis 51. At this time, the trigger member 600 may press the clutch unit 500 to couple the rod 410 and the driving wheel 420. In an embodiment, the trigger member 600 may release the contact with the starter 650, and the starter 650 may press the driving shaft 330 to move the position of the contact member 350 located inside the driving module 300. The trigger member 600 may be divided into a plurality of bent parts.

The trigger member 600 may have the first end 610 extending to the needle assembly 100. The first end 610 is disposed to be in contact with the first stopper 52, and when the user rotates the needle assembly 100, the knob 101 may press a first flange 611 to start the rotation of the trigger member 600. After the first end 610 rotates, a cut-out groove 612 is supported by the side wall 52b of the first stopper 52 to limit the opposite-direction rotation of the trigger member 600.

The trigger member 600 may have the second end 620 extending to the clutch unit 500. The second end 620 may extend in a direction which is different from the direction in which the first end 610 extends, and may include a second flange 621 that is to be inserted between the end 511 and the driving wheel 420. When the trigger member 600 rotates, the second flange 621 presses the end 511 of the coupler 510 so that the coupler 510 is engaged with the inserter 520, thereby activating the clutch unit 500.

The trigger member 600 may include a third end 630 extending to the driving module 300. The third end 630 may extend toward the driving module 300 and may start driving the driving module 300.

The third end 630 may include a support piece 631 and a guide piece 632 which are bent toward the driving shaft 330. As illustrated in FIGS. 5A and 6, the support piece 631 may support the starter 650 before driving the drug injection device 1. The guide piece 632 may be arranged adjacent to the support piece 631 and may support or move along the sidewall of the moving unit 340.

The starter 650 maintains the driving shaft of the driving module 300 in a fixed state by the third end 630, but when the trigger member 600 rotates, the contact between the third end 630 and the starter 650 is released, and the starter 650 presses the drive shaft to start the driving of the driving module 300.

The starter 650 may move the driving module 300 by the rotation of the trigger member 600. When the contact with the trigger member 600 is released, the starter 650 may press a moving unit 340 of the driving module 300 to move the driving shaft.

In an embodiment, the starter 650 may be formed in the form of a spring having a preset elasticity, but is not limited thereto and may be formed in a stick form having a predetermined elasticity.

An end of the starter 650 is mounted on the base body 50, and another end thereof is arranged to support an end of the trigger member 600. The end of the starter 650 is disposed between the end of the drive shaft and the third end 630 of the trigger member 600.

The starter 650 may press the end of the driving shaft 330 by the rotation of the trigger member 600. Before the drug injection device 1 is driven, the starter 650 maintains a compression state. However, when the contact thereof with the support piece 631 is released, a pressing end 651 presses the moving unit 340 by the expansion force of the starter 650. In this case, the driving shaft 330 connected to the moving unit 340 and the contact member 350 provided on the driving shaft 330 may be moved according to the movement of the moving part. Thereafter, an electrochemical reaction is initiated in the driving module 300, and driving of the driving module 300 is started.

The battery 700 may supply power to the drug injection device 1. The driving module 300 may be connected to the battery 700 to control the driving of the driving module 300. The battery 700 may be rechargeable or may be used for single use.

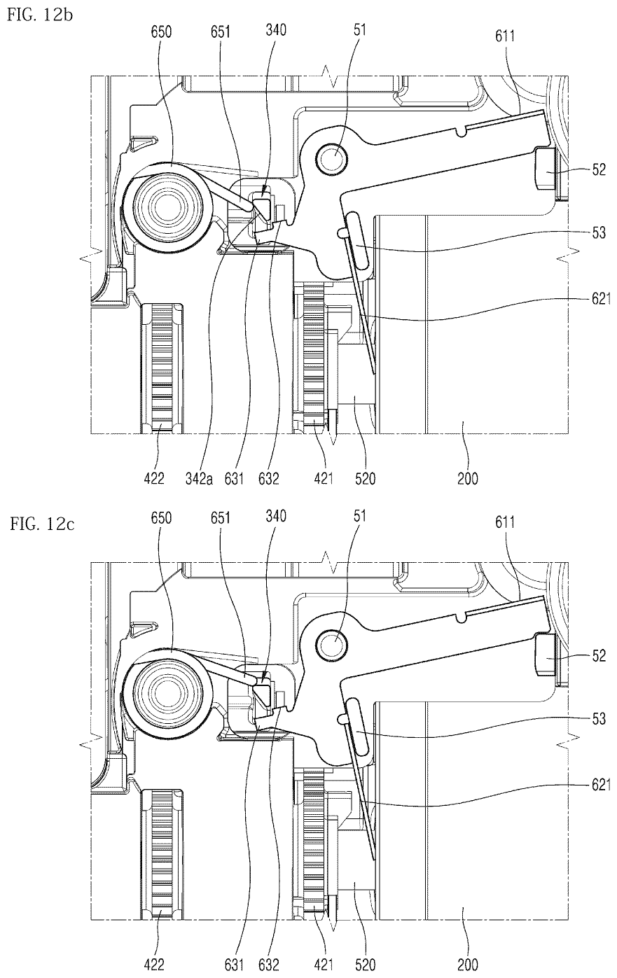

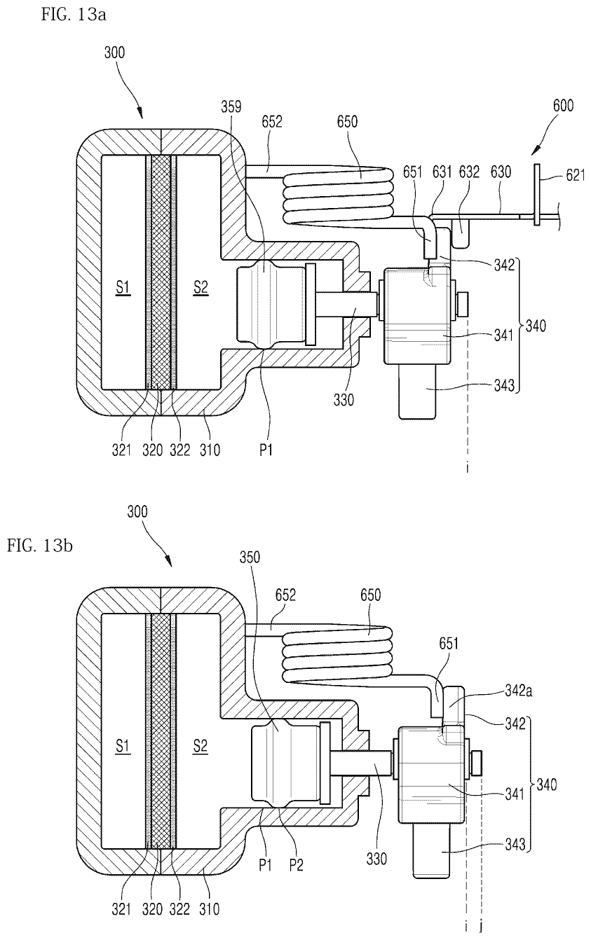

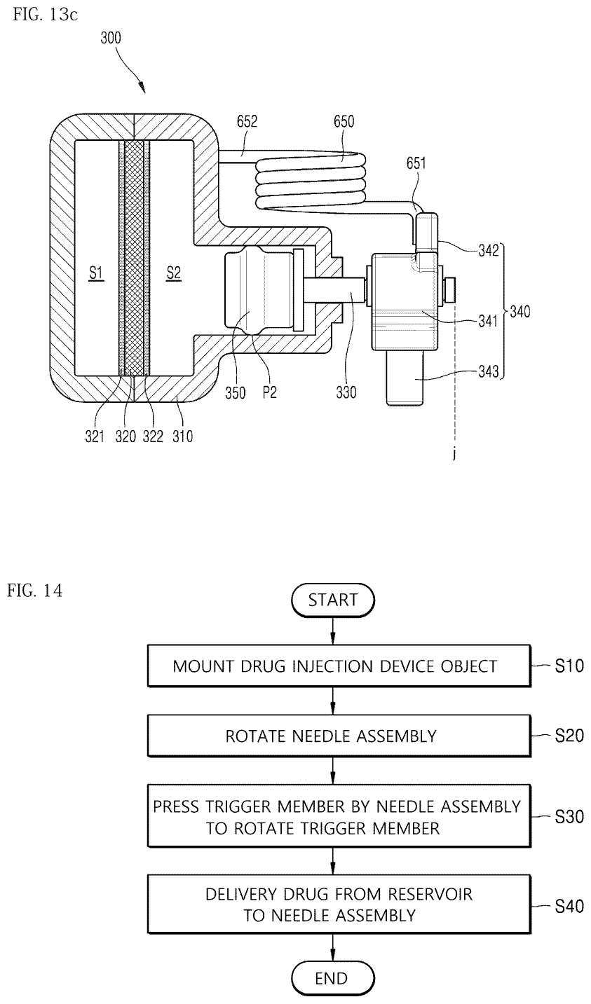

FIGS. 12A to 12C are diagrams illustrating the driving of the driving module 300 according to the rotation of the trigger member 600 of FIG. 2 from one side. FIGS. 13A to 13C are diagrams illustrating the driving of a driving module 300 according to the rotation of the trigger member 600 of FIG. 2 from another side.

Referring to FIGS. 12A and 13A, the support piece 631 of the trigger member 600 maintains the compression state of the starter 650. The starter 650 is mounted on the base body 50 in the preset compression state, and since the support piece 631 retains the fixed position, the compression force is not released. The support piece 631 of the trigger member 600 remains in contact with the pressing end 651 of the starter 650, limiting the movement of the starter 650.

The contact member 350 is located at a first position P1 on the inner surface of the cover 310, and the end of the driving shaft 330 is located at position i. The first position P1 is defined as a position where the contact member 350 contacts the inner surface of the cover 310 before the drug injection device 1 is driven, and is not limited to a specific position. Since no external force is transmitted to the driving shaft 330, the contact member 350 is located in the preset first position P1.

At this time, since the clutch unit 500 is not driven, the coupler 510 is not connected to the inserter 520. Therefore, the rod 410 of the driving unit 400 and the driving wheel 420 are in a drivably separated state, and the drug stored in the reservoir 200 does not flow into the needle N.

Referring to FIGS. 12B and 13B, the trigger member 600 rotates to release the contact between the support piece 631 and the starter 650. When the knob 101 of the needle assembly 100 presses the first flange 611, the trigger member 600 rotates about the rotation axis 51, and the support piece 631 also rotates away from the moving unit 340. At this time, the contact between the support piece 631 and the pressing end 651 is released.

At the same time, the pressing end 651 presses the moving unit 340 to move the driving shaft 330. As the pressing force is released, the pressing end 651 comes into contact with the inclined surface 342a and moves along the inclined surface 342a. The pressing end 651 axially pushes the moving unit 340, so that the moving unit 340 and the driving shaft 330 move away from the driving module 300. The end of the driving shaft 330 moves from position i to position j, and the contact member 350 moves together with the driving shaft 330 to be positioned at the second position P2. At this time, the clutch unit 500 is driven to couple the coupler 510 with the inserter 520. Since the end 511 of the coupler 510 is separated from the driving wheel 420 and grips the outer side of the inserter 520, the driving wheel 420, the coupler 510, the inserter 520, and the rod 410 are driven as one body.

Referring to FIGS. 12C and 13C, the pressing end 651 moves beyond the inclined surface 342a. Based on FIG. 5A, the pressing end 651 is positioned on the opposite side of the moving unit 340, and the contact thereof with the moving unit 340 is released. The moving unit 340 may be supported on one side of the base body 50.

In this case, an electrochemical reaction may occur in the driving module 300, and a driving force for linearly reciprocating the driving shaft 330 may be generated. The result of the electrochemical reaction occurring inside the driving module 300 causes a volume change in the first space S1 and the second space S2. This volume change is transmitted to the driving shaft 330 as a driving force. That is, when the volume of the second space S2 is increased, the driving shaft 330 moves forward, and when the volume of the second space S2 is decreased, the driving shaft 330 moves backward. The movement of the driving shaft 330 linearly reciprocates the moving unit 340, and the connection shaft 343 of the moving unit 340 rotates the driving wheel 420 toward a connector (not shown) to discharge the drug from the reservoir 200.

After the drug injection device 1 is manufactured and before being driven, the contact member 350 may be in contact with a specific area (first position) of the cover 310 for a long time. The contact member 350 may include a silicone-based or rubber-based material. Accordingly, the contact member 350 may be deformed due to prolonged contact and attached on the inner surface of the cover 310. When the contact member 350 is attached on the inner surface of the driving module 300, the contact member 350 may interfere with the initial driving of the driving module 300, causing a difficulty in discharging the drug in a preset amount, from the needle N.

The starter 650 may move the contact member 350 before driving the drug injection device 1, so that the driving module 300 may have an optimized driving environment. In detail, when the contact between the starter 650 and the trigger member 600 is released, the starter 650 moves the driving shaft 330 of the driving module 300. Accordingly, the contact position of the contact member 350 may be changed before the driving module 300 is driven by an electrochemical reaction. Since the starter 650 moves the position of the contact member 350 from the first position P1 to a second position P2, the attachment and fixation of the contact member 350 on the inner surface of the driving module 300 is eliminated, and the initial driving of the driving module 300 may proceed smoothly.

The drug injection device 1 according to an embodiment of the present disclosure may be driven simply and safely by the user. When the user rotates the sleeve 110 of the needle assembly 100, the cannula of the needle assembly 100 is inserted into the object and the clutch unit 500 engages the driving unit with the driving unit 400 to allow the drug to be injected due to the driving of the driving module 300, and at the same time, the driving of the driving module 300 is started. Accordingly, the drug injection device 1 can be used simply and safely.

The drug injection device 1 according to an embodiment of the present disclosure may discharge the drug in a preset amount since the driving module 300 is driven in an optimized driving environment. The starter 650 may move the position of the driving shaft 330 before the driving module 300 is driven, so that the case in which the contact member 350 is attached on the inner surface of the driving module 300 to prevent the driving of the driving module 300, may be prevented. Since the storability of the drug injection device 1 is improved and precision thereof is maintained, even when the drug injection device 1 is driven after being stored for a long time, the driving module 300 may be operated smoothly at the beginning, so that the drug can be discharged in a preset amount to the object.

In relation to the drug injection device 1 according to an embodiment of the present disclosure, after the cannula C of the needle assembly 100 is inserted into the object, the structure for discharging the drug is drivably engaged by the clutch unit 500. Accordingly, the drug may be stably injected in a preset amount into the object.

The driving of the drug injection device 1 according to an embodiment of the present disclosure is intuitively started by rotation of the needle assembly 100. Also, since the rotation directions of the needle assembly 100 and the trigger member 600 are limited to one direction, the rotation distance is limited to a preset distance. Accordingly, user safety can be secured.

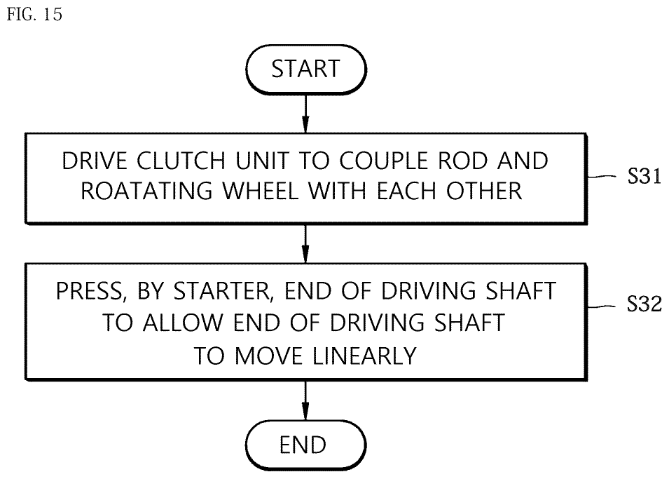

FIG. 14 is a flowchart illustrating a method of driving a drug injection device according to another embodiment of the present disclosure, and FIG. 15 is a flowchart illustrating some operations of FIG. 14.

Referring to FIGS. 14 to 15, the method of driving a drug injection device 1 includes mounting the drug injection device on an object (S10), rotating a needle assembly 100 (S20), and pressing a trigger member by the needle assembly to rotate the trigger member (S30), and delivering the drug from a reservoir to the needle assembly (S40).

In the mounting the drug injection device on an object (S10), the attachment portion 6 of the drug injection device 1 is attached to the object. In addition, the drug injection device 1 may be connected with the remote device 2 in a wired/wireless communication environment.

In the rotating the needle assembly 100 (S20), the needle assembly 100 may be driven by the user. As described above, when the sleeve 110 of the needle assembly 100 is rotated first by the user, the cannula C and the needle N are inserted into the skin together (see FIG. 5B), and when the sleeve 110 is rotated again, only the needle N is withdrawn from the skin (see FIG. 5C). The first rotation of the sleeve 110 is to fix the cannula C to the skin using the needle N, and the second rotation is to withdraw the needle N and inject the drug.

The process in which the trigger member is pressed by the needle assembly to rotate the trigger member (S30), occurs at the same time when the needle assembly 100 rotates. When the needle assembly 100 rotates, the knob 101 presses the first flange 611 of the trigger member 600.

At this time, the operation (S31) in which the rod and the rotary wheel are coupled by driving the clutch mechanism and the operation (S32) in which the starter presses the end of the drive shaft to move the end of the drive shaft linearly, may occur substantially simultaneously.

In the operation (S31) in which the rod and the rotating wheel are coupled by driving the clutch unit, the second flange 621 of the trigger member 600 presses the end 511 of the coupler 510 to activate the clutch unit 500. As illustrated in FIG. 9, the coupler 510 engages the rod 410 with the driving wheel 420, so that a driving force generated by the driving module 300 may be transmitted to the plunger 230 through the driving unit 400.

In the operation (S31) in which the starter presses the end of the driving shaft and the end of the drive shaft moves linearly, when the contact between the starter 650 and the third end 630 is released, the starter 650 may start driving of the driving module 300. As the starter 650 presses and passes through the end of the driving shaft, the end of the driving shaft moves linearly along the axis. Thereafter, the driving module 300 may linearly move the driving shaft repeatedly, so that a driving force may be transmitted to the driving unit 400.

In the delivering of the drug from the reservoir to the needle assembly (S40), an electrochemical reaction occurs in the driving module 300, and the drug in the reservoir 200 may be delivered to the object by repetitive linear movement of the driving shaft of the driving module.

The method of driving the drug injection device according to an embodiment of the present disclosure may be driven simply and safely by the user. When the user rotates the sleeve 110 of the needle assembly 100, the cannula of the needle assembly 100 is inserted into the object, and the clutch unit 500 engages the driving unit 400, so that due to the driving of the driving module 300, the drug can be injected and at the same time, the driving of the driving module 300 is started. Accordingly, the drug injection device 1 can be used simply and safely.

The method of driving the drug injection device according to an embodiment of the present disclosure may discharge the drug in a preset amount since the driving module 300 is driven in an optimized driving environment. The starter 650 may move the position of the driving shaft 330 before the driving module 300 is driven, so that the case in which the contact member 350 is attached on the inner surface of the driving module 300 to prevent the driving of the driving module 300, may be prevented. Since the storability of the drug injection device 1 is improved and precision thereof is maintained, even when the drug injection device 1 is driven after being stored for a long time, the driving module 300 may be operated smoothly at the beginning, so that the drug can be discharged in a preset amount to the object.

In relation to the method of driving the drug injection device according to an embodiment of the present disclosure, after the cannula C of the needle assembly 100 is inserted into the object, the structure for discharging the drug is drivably engaged by the clutch unit 500. Accordingly, the drug may be stably injected in a preset amount into the object.

Regarding the method of driving the drug injection device according to an embodiment of the present disclosure, the driving of the drug injection device 1 is intuitively started by rotation of the needle assembly 100. Also, since the rotation directions of the needle assembly 100 and the trigger member 600 are limited to one direction, the rotation distance is limited to a preset distance. Accordingly, user safety can be secured.

Therefore, the concept of the present disclosure should not be limited to the above-described embodiments, and not only the claims described below, but also all scopes equivalent to or equivalently changed from these claims are included in the scope of the concept of the present disclosure.

### INDUSTRIAL APPLICABILITY

According to an embodiment of the present disclosure, the drug injection device may be applied to various industrially available devices. The drug injection device can be applied to devices that deliver various drugs.

1. A drug injection device comprising:
a base body;
a needle assembly mounted on the base body;
a reservoir mounted on the base body and fluidly connected with the needle assembly;
a driving unit including a rod configured to engage with a plunger disposed inside the reservoir and configured to move along the reservoir, and a driving wheel configured to transmit a driving force to the rod;
a clutch unit disposed between the rod and the driving wheel; and
a trigger member rotatably disposed on one side of the base body and coupling the rod with the driving wheel by pressing the clutch unit.

2. The drug injection device of claim 1, wherein
the needle assembly is configured to rotate in one direction to press an end of the trigger member so as to rotate the trigger member, such that another end of the trigger member presses a coupler of the clutch unit so that an end of the coupler is separated from the driving wheel.

3. The drug injection device of claim 1, wherein
the trigger member is configured to press the clutch unit after a cannula of the needle assembly is inserted.

4. The drug injection device of claim 1, wherein
the base body is provided with a stopper for limiting the rotation distance of the trigger member.

5. The drug injection device of claim 1, wherein the clutch unit comprises:
an inserter inserted into the rod; and
a coupler disposed on the outside of the inserter, and having an end that is detachably mounted on the driving wheel.

6. The drug injection device of claim 5, wherein the driving wheel comprises:
a first supporter disposed on one surface of the driving wheel to support an end of the coupler so as to maintain a state in which the diameter of the coupler is expanded; and
a second supporter disposed to be spaced apart from the first supporter on the surface of the driving wheel and supporting the end of the coupler so as to maintain a state in which the diameter of the coupler is contracted.
